(19) United States
(12) Patent Application Publication
(10) Pub. No.: US 2025/0064421 A1
(43) Pub. Date: Feb. 27, 2025

(54) CALIBRATION DEVICE FOR MEDICAL IMAGING SYSTEMS

(71) Applicant: SAVFE Co., Ltd., Taipei (TW)

(72) Inventors: HAO-KAI CHOU, Taipei (TW); CHIH-MIN YANG, Taipei (TW)

(21) Appl. No.: 18/816,479

(22) Filed: Aug. 27, 2024

Related U.S. Application Data
(60) Provisional application No. 63/534,845, filed on Aug. 27, 2023.

(51) Int. Cl. A61B 6/58

(52) U.S. Cl. CPC A61B 6/582

(57) ABSTRACT

A calibration device for a medical imaging system is provided. The calibration device includes a structural framework and multiple markers arranged on the structural framework. At least one of the following two conditions are satisfied: at least two markers are with different absorption rates for a radiation used by the medical imaging system; and at least two positions within one of the markers are with different absorption rates for the radiation.

### CROSS-REFERENCE TO RELATED APPLICATION(S)

The present disclosure claims the benefit of and priority to U.S. Provisional Patent Application Ser. No. 63/534,845, filed on Aug. 27, 2023, entitled “X-RAY SIMULATION SYSTEM,” the content of which is hereby incorporated herein fully by reference into the present disclosure for all purposes.

### FIELD

The present disclosure generally relates to surgical assistance technology and, more particularly, to a calibration device for medical imaging systems.

### BACKGROUND

In surgical procedures, accurately locating a lesion often relies heavily on the experience of the surgeon. For instance, during a minimally invasive orthopedic surgery, a surgeon frequently needs to take multiple X-ray images to verify a spatial position of surgical instruments throughout the procedure. However, the need for multiple X-ray images exposes both the surgeon and the patient to excessive radiation doses, and repeated imaging processes also significantly increases the duration of the surgery. With advancements in technology, various surgical navigation systems have been developed to replace the need for multiple X-ray images during a surgery. Nevertheless, existing surgical navigation systems depend on additional spatial sensing devices, such as infrared optical sensors or electromagnetic field sensors, for spatial positioning. Typically, the use of such external spatial sensing devices for instrument navigation not only requires the sensing devices themselves but also necessitates equipping each surgical tool and the patient with detectable components, which results in added complexity within the surgical environment, increased costs, and longer preparation times for the surgical navigation system. Moreover, during the operation, issues such as occlusion or electromagnetic interference may lead to instability in the spatial sensing devices, thus further complicating the procedure.

### SUMMARY

The present disclosure is directed to a calibration device, which may be use with a medical imaging system and helpful for accurate surgical tool guidance using the medical imaging system.

According to the present disclosure, a calibration device for a medical imaging system is provided. The calibration device includes a structural framework and multiple markers arranged on the structural framework. At least one of the following two conditions are satisfied: at least two markers are with different absorption rates for a radiation used by the medical imaging system; and at least two positions within one of the markers are with different absorption rates for the radiation.

In some implementations of the present disclosure, a number of the markers is greater than or equal to 4.

In some implementations of the present disclosure, one of the markers includes a multilayer structure, the multilayer structure includes at least one imaging layer, and a number of the at least one imaging layer is adjustable in the multilayer structure.

In some implementations of the present disclosure, one of the markers includes a first sphere and a second sphere, the second sphere is embedded in the first sphere, and the first sphere and the second sphere have different absorption rates for the radiation.

In some implementations of the present disclosure, one of the markers includes a first sphere and a second sphere, and the second sphere forms a hollow interior of the first sphere.

In some implementations of the present disclosure, one of the markers includes a housing and a gate. The housing is at least partially transparent for the radiation, and the gate is configured to allow a contrast agent to flow into the housing.

In some implementations of the present disclosure, the markers include a grid with multiple cells, and adjacent cells of the multiple cells have different absorption rates for the radiation.

In some implementations of the present disclosure, a first marker includes a protrusion structure on the structural framework, and a second marker includes a recessed structure on the structural framework.

In some implementations of the present disclosure, the structural framework includes a first ring located on a first plane, and a second ring located on a second plane parallel to the first plane. At least three markers are located at the first ring and at least three markers are located at the second ring.

In some implementations of the present disclosure, the second ring is concentric with the first ring.

In some implementations of the present disclosure, the first ring and the second ring define a hollow circular frustum or a hollow cylinder.

In some implementations of the present disclosure, at least one marker is non-coplanar with the other markers.

In some implementations of the present disclosure, the radiation includes an X-ray.

### BRIEF DESCRIPTION OF THE DRAWINGS

Aspects of the example disclosure are best understood from the following detailed description when read with the accompanying figures. Various features are not drawn to scale. Dimensions of various features may be arbitrarily increased or reduced for clarity of discussion.

FIG. 1 is a block diagram illustrating a surgical system, according to an example implementation of the present disclosure.

FIG. 2 is a diagram illustrating a surgical system, according to an example implementation of the present disclosure.

FIG. 3 is a diagram illustrating an arm, according to an example implementation of the present disclosure.

FIG. 4A to FIG. 4E are diagrams illustrating a configuration of arm joint(s) using a magnetic encoder, according to example implementations of the present disclosure.

FIG. 5A to FIG. 5D are diagrams illustrating a configuration of arm joint(s) using a capacitive encoder, according to example implementations of the present disclosure.

FIG. 6 is a diagram illustrating a terminal adapter structure, according to an example implementation of the present disclosure.

FIG. 7 is a diagram illustrating a structure configured to connect/secure/limit the instrument, according to an example implementation of the present disclosure.

FIG. 8 is a diagram illustrating a determination of positions of the markers, according to an example implementation of the present disclosure.

FIG. 9 is a diagram illustrating a determination of a position of an instrument, according to an example implementation of the present disclosure.

FIG. 10 is a diagram illustrating a calibration device, according to an example implementation of the present disclosure.

FIG. 11A to FIG. 11E are diagrams illustrating markers on the calibration device, according to example implementations of the present disclosure.

FIG. 12 is a flowchart illustrating a method/process for navigating a surgical operation, according to an example implementation of the present disclosure.

FIG. 13 is a diagram illustrating an output image, according to an example implementation of the present disclosure.

### DETAILED DESCRIPTION

The following description contains specific information pertaining to exemplary implementations in the present disclosure. The drawings in the present disclosure and their accompanying detailed description are directed to merely exemplary implementations. However, the present disclosure is not limited to merely these exemplary implementations. Other variations and implementations of the present disclosure will occur to those skilled in the art. Unless noted otherwise, like or corresponding elements among the figures may be indicated by like or corresponding reference numerals. Moreover, the drawings and illustrations in the present disclosure are generally not to scale, and are not intended to correspond to actual relative dimensions.

For the purpose of consistency and ease of understanding, like features are identified (although, in some examples, not shown) by numerals in the example figures. However, the features in different implementations may be differed in other respects, and thus shall not be narrowly confined to what is shown in the figures.

References to “one implementation,” “an implementation,” “example implementation,” “various implementations,” “some implementations,” “implementations of the present application,” etc., may indicate that the implementation(s) of the present application so described may include a particular feature, structure, or characteristic, but not every possible implementation of the present application necessarily includes the particular feature, structure, or characteristic. Further, repeated use of the phrase “in one implementation,” or “in an example implementation,” “an implementation,” do not necessarily refer to the same implementation, although they may. Moreover, any use of phrases like “implementations” in connection with “the present application” are never meant to characterize that all implementations of the present application must include the particular feature, structure, or characteristic, and should instead be understood to mean “at least some implementations of the present application” includes the stated particular feature, structure, or characteristic. The term “coupled” is defined as connected, whether directly or indirectly through intervening components, and is not necessarily limited to physical connections. The term “comprising,” when utilized, means “including, but not necessarily limited to”; it specifically indicates open-ended inclusion or membership in the so-described combination, group, series and the equivalent.

Additionally, for the purposes of explanation and non-limitation, specific details, such as functional entities, techniques, protocols, standard, and the like are set forth for providing an understanding of the described technology. In other examples, detailed description of well-known methods, technologies, system, architectures, and the like are omitted so as not to obscure the description with unnecessary details.

The terms “first,” “second,” and “third,” etc. used in the specification and the accompanying drawings of the present disclosure are intended to distinguish between different objects, rather than to describe a particular order. In addition, the term “comprising” and any variations thereof are intended to cover non-exclusive inclusion. For example, a process, method, system, product, or apparatus that includes a series of steps or modules is not limited to the listed steps or modules but may optionally include additional steps or modules not listed, or optionally include additional steps or modules inherent in such processes, methods, products, or apparatus.

The following description is provided in conjunction with the accompanying drawings to illustrate implementations of the present disclosure.

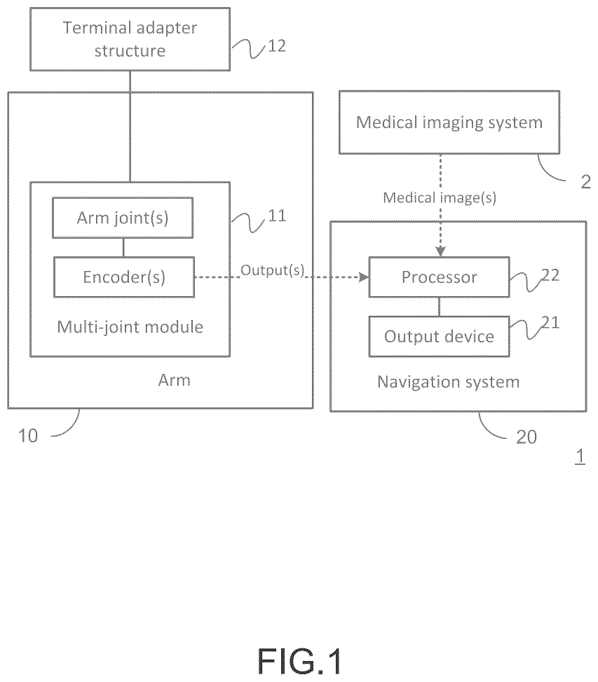

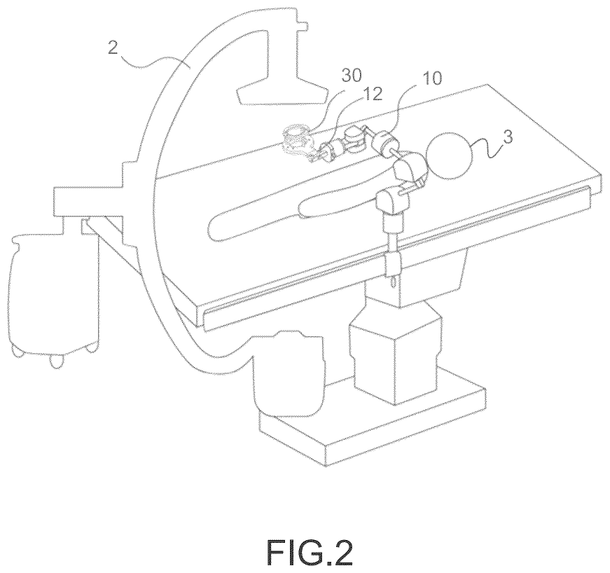

FIG. 1 is a block diagram illustrating a surgical system, according to an example implementation of the present disclosure. FIG. 2 is a diagram illustrating a surgical system, according to an example implementation of the present disclosure.

Referring to FIG. 1 and FIG. 2, a surgical system 1 may be utilized to perform (surgical) operations on a patient 3. The surgical system 1 may include at least one arm 10 and a navigation system 20. The navigation system 20 may include an output device 21 and a processor 22, and the processor 22 may be coupled to the output device 21 and obtain medical images captured by a medical imaging system 2. The at least one arm 10 may be, for example, a mechanical arm.

In some implementations, the medical imaging system 2 may be an X-ray imaging system. The X-ray imaging system may include C-Arm, Computed Tomography (CT), 3D C-Arm, O-Arm, handheld X-ray imaging devices, surgical tables with integrated X-ray sources, and/or other equipment with X-ray radiation sources.

In some implementations, the medical imaging system may be a magnetic resonance imaging (MRI) system or a positron emission tomography (PET) system.

In some implementations, the processor 22 may be coupled to the medical imaging system 2. After capturing medical image(s) using the medical imaging system 2, the medical image(s) may be obtained by the processor 22.

In some implementations, the processor 22 may be coupled to a device with photographic capabilities, and may obtain the medical image(s) through a reshooting process. Specifically, after capturing the medical image(s) using the medical imaging system 2 and obtaining capturing outputs, the device with photographic capabilities may capture the capturing outputs of the medical imaging system 2 for importing to the processor 22. The device with photographic capabilities may include, for example, a mobile phone and/or a camera. The importing may be performed via various transmission methods including, for example, wired interfaces, such as Type C, Universal Serial Bus (USB); or wireless transmission methods, such as Wi-Fi and Bluetooth.

It should be noted that, any function(s) or algorithm(s) performed by the processor 22 may be implemented by software, hardware, firmware, or any combination thereof. In some examples, the software implementation may include computer-executable instructions stored on a (e.g., non-transitory) computer-readable medium such as memory or other types of storage devices.

In some implementations, the output device 21 may be configured to present information of the navigation system 20 to the user, for example, in a visual format (e.g., a 2D/3D image). In some implementations, the information may include simulated image(s) generated by the navigation system 20. In some implementations, the information may include at least one of the following: the medical image(s) captured by the medical imaging system 2, surgical instruction contour image(s), simulated medical imaging equipment image(s), the Field of View (FOV) of the simulated medical imaging equipment, guidance prompts provided to the user, and/or a combination/superposition of those described above.

In some implementations, the output device 21 may include at least one monitor positioned according to the user's personal preference. In addition, the at least one monitor may take the form of a 360-degree circular screen or projected images, such as Light Field 3D, Floating Pictogram Technology (FPT), floating virtual screens, holographic projections, and so on. In some implementations, the output device 21 may include a head-mounted device that uses VR (Virtual Reality), AR (Augmented Reality), or MR (Mixed Reality) methods.

In some implementations, the arm 10 may include a multi-joint module 11. The multi-joint module 11 may include, for example, multiple arm joints, at least one connecting shaft, and an adapter interface, when needed. The at least one connecting shaft may be configured to connect the arm joints, while the adapter interface may be configured to connect and secure the arm 10 to a fixing device, such as a height adjustment device, side rail clamp, or workstation, as shown in FIG. 2.

Referring to FIG. 2, in some implementations, the multi-joint module 11 may further include an end effector module, where the end effector module may be configured to directly or indirectly connect the terminal adapter structure 12 which is for connecting tools such as a calibration device 30 or an instrument (which will be described later). In some examples, at least one tool may be connected between the end effector module and the terminal adapter structure 12. In some examples, the end effector module may connect the terminal adapter structure 12. In some implementations, the end effector module may include the terminal adapter structure 12.

In some implementations, the multi-joint module 11 may further include one or more encoders, such that each of the arm joints may correspond to an encoder for acquiring moving/rotating information of the corresponding arm joint.

In some implementations, all or at least a part of the arm joints may be a rotary joint, which may be characterized by having at least one degree of freedom. In some implementations, to achieve better operational flexibility, the arm 10 may be equipped with at least six degrees of freedom though a combination of the arm joints.

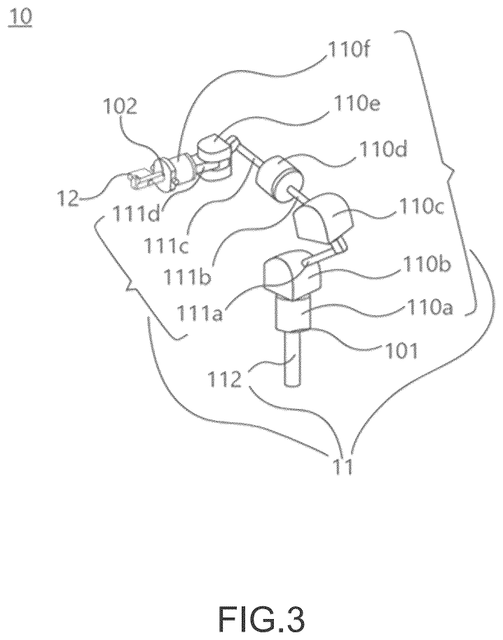

FIG. 3 is a diagram illustrating an arm, according to an example implementation of the present disclosure.

Referring to FIG. 3, in some implementations, the arm 10 may include a multi-joint module 11, which may include multiple arm joints 110a, 110b, 110c, 110d, 110e; an end effector module 110f; multiple connecting shafts 111a, 111b, 111c, 111d; and an adapter interface 112. Each of the arm joints 110a, 110b, 110c, 110d, 110e may connect/include/correspond to an encoder (not shown in FIG. 3), and information sensed by the encoders may be used to calculate three-dimensional (3D) coordinate system information of the arm 10. The 3D coordinate system information of the arm 10 may include the six degrees of freedom information for each endpoint of the arm 10. The endpoints may include at least one of a base end 101 of the arm 10, each of the arm joints 110a, 110b, 110c, 110d, 110e, the end effector module 110f, and a terminal end 102 of the arm 10. In some examples, the terminal end 102 may be located on the end effector module 110f. The arm 10 may be a passive joint arm or an active arm. In some implementations, the end effector module 110f may connect/include/correspond to an encoder as well. In some implementations, the end effector module 110f may be considered to be one of the arm joints.

In some implementations, the encoders corresponding to the arm joints may be coupled to the processor 22 of the navigation system 20, such that the processor 22 may obtain the outputs of the encoders. In some example implementations, all the encoders may be coupled to a controller (e.g., located at the base of the arm 10) of the arm 10, and the processor 22 may be coupled to the controller.

In some implementations, each encoder may be a magnetic encoder, a capacitive encoder, and/or an optical encoder. The magnetic encoder may be, for example, a non-contact magnetic encoder. In some implementations, each encoder is connected to an output shaft of the corresponding arm joint to reduce measurement errors that are caused by the transmission mechanism. In some implementations, all components within each arm joint are coaxially assembled.

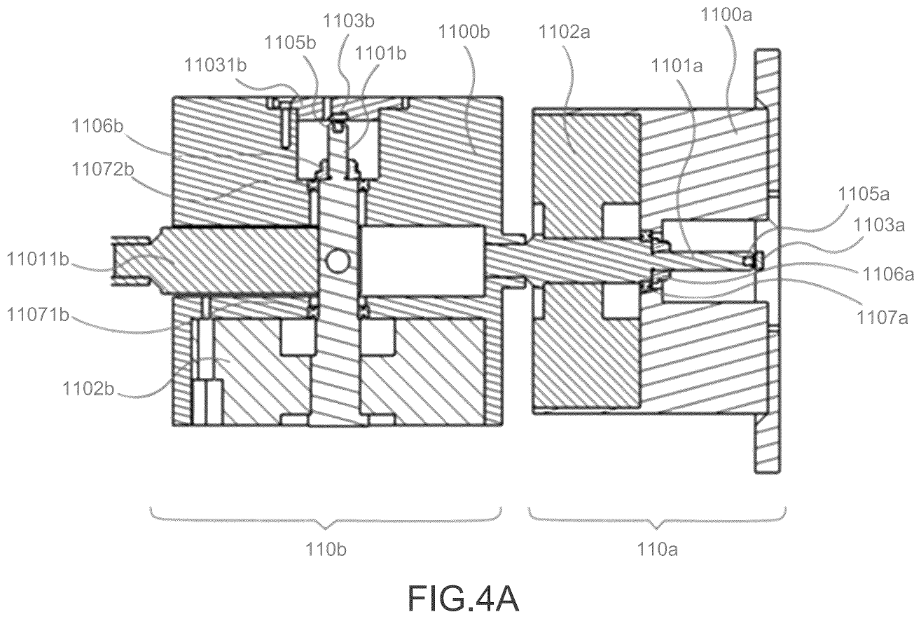

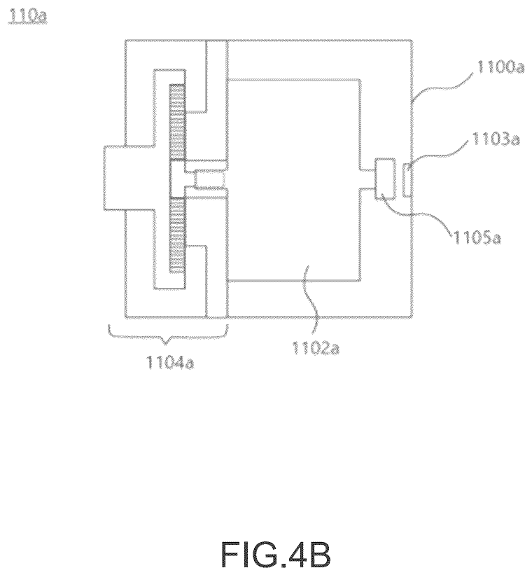

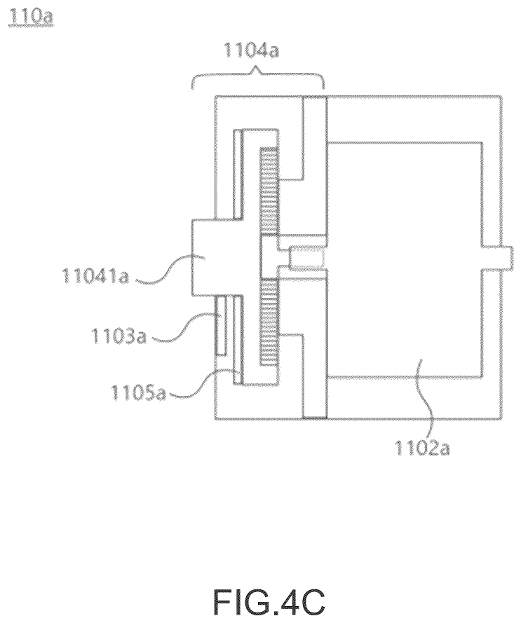

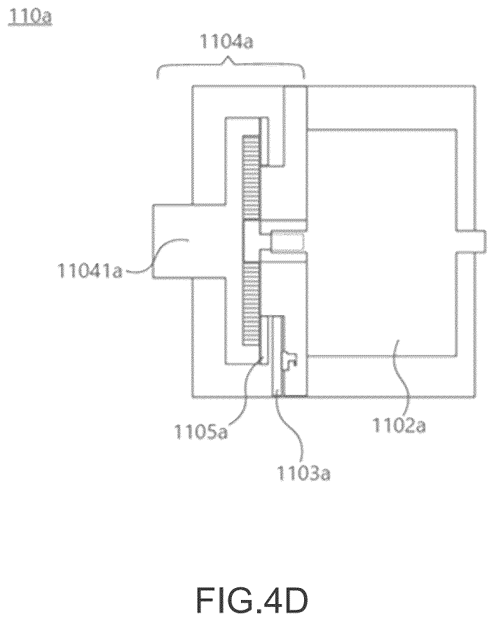

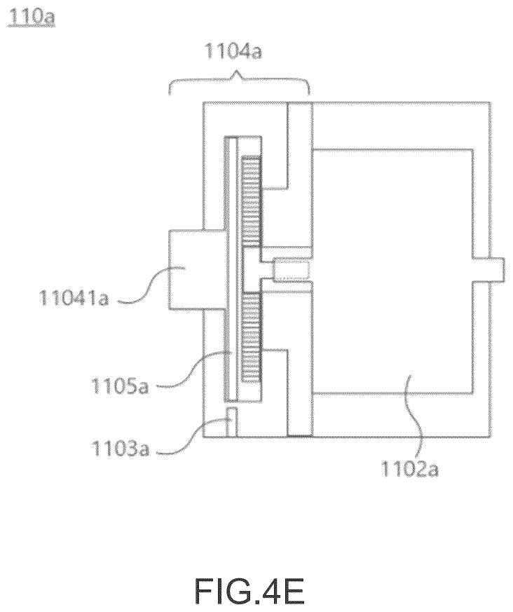

FIG. 4A to FIG. 4E are diagrams illustrating a configuration of arm joint(s) using a magnetic encoder, according to example implementations of the present disclosure. In implementations described with reference to FIGS. 4A to 4E, arm joints 110a and 110b are used as examples for description purposes. However, it should be understood that the descriptions do not limit the specific positions of the arm joints 110a and 110b within the arm 10. The arm joint 110a and the arm joint 110b may be, for example, rotary joints.

Referring to FIG. 4A, in some implementations, the arm joint 110a may include a base 1100a that supports the internal components of the arm joint 110a. The magnetic encoder 1103a may be used in conjunction with the magnet 1105a, and the magnet 1105a may be connected to the rotating shaft 1101a. Additionally, the arm joint 110a may include a bearing 1107a that is sleeved onto the rotating shaft 1101a and a nut 1106a. The bearing 1107a may support the rotating shaft 1101a and counteract radial forces, to ensure the rotational movement of the rotating shaft 1101a. The nut 1106a may secure the rotating shaft 1101a to prevent the rotating shaft 1101a from dislodging from the bearing 1107a. The rotating shaft 1101a, brake 1102a, magnet 1105a, and encoder 1103a within the arm joint 110a may be, for example, coaxially assembled.

The arm joint 110b may include a base 1100b that supports the internal components of the arm joint 110b and a fixture 11031b for securing the magnetic encoder 1103b. The magnetic encoder 1103b may be used in conjunction with the magnet 1105b, and the magnet 1105b may be connected to the rotating shaft 1101b. Additionally, the rotary joint 110b may include bearings 11071b and 11072b that are sleeved onto the rotating shaft 1101b and a nut 1106b. The bearings 11071b and 11072b may support the rotating shaft 1101b and counteract radial forces to ensure the rotational movement of the rotating shaft 1101b. The nut 1106b may secure the rotating shaft 1101b to prevent the rotating shaft 1101b from dislodging from the bearing 11072b. The rotating shaft 1101b, brake 1102b, magnet 1105b, and encoder 1103b within the arm joint 110b may be, for example, coaxially assembled.

Referring to FIG. 4B, in some implementations, the arm joint may include a reducer for torque amplification. The arm joint 110a may include a reducer 1104a that is connected to one side of the brake 1102a, while the magnet 1105a may be connected to the opposite side of the brake 1102a. The magnetic encoder 1103a may be positioned relative to the magnet 1105a and be fixed to the base 1100a of the arm joint 110a.

In some implementations, the magnet within the arm joint may be a magnetic ring.

Referring to FIG. 4C, in some implementations, the arm joint 110a may include a magnetic ring 1105a that is sleeved onto the output shaft end 11041a of the reducer 1104a, and the magnetic encoder 1103a may be positioned relative to the magnetic ring 1105a. The magnetic ring 1105a may be sleeved onto the output shaft end 11041a of the reducer 1104a, placed on the side of the reducer 1104a opposite the output shaft end 11041a, or positioned on the side of the reducer 1104a. The magnetic encoder 1103a may be positioned relative to the magnetic ring 1105a. Advantageously, this configuration may save internal space within the arm joint and keep the magnetic ring 1105a away from the electromagnetic brake 1102a, thus avoiding magnetic interference.

Referring to FIG. 4D, in some implementations, the magnetic ring 1105a may be placed on the side of the reducer 1104a opposite the output shaft end 11041a. Referring to FIG. 4E, in some implementations, the magnetic ring 1105a may be positioned on the side of the reducer 1104a.

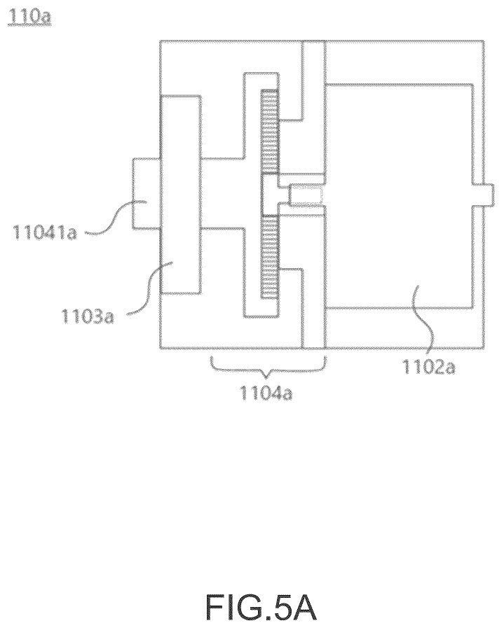

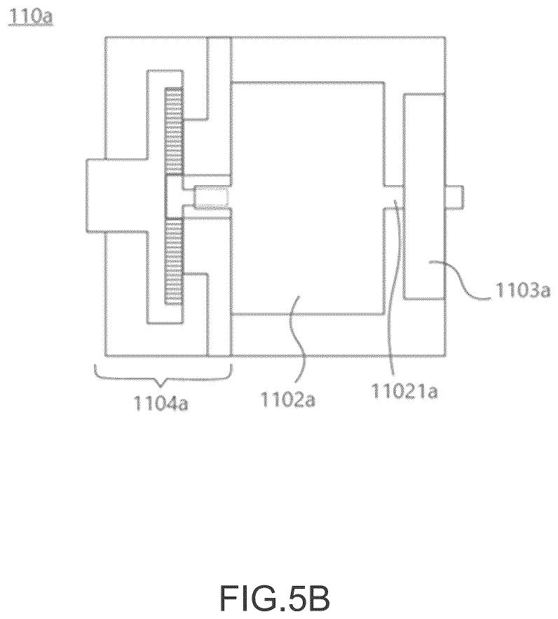

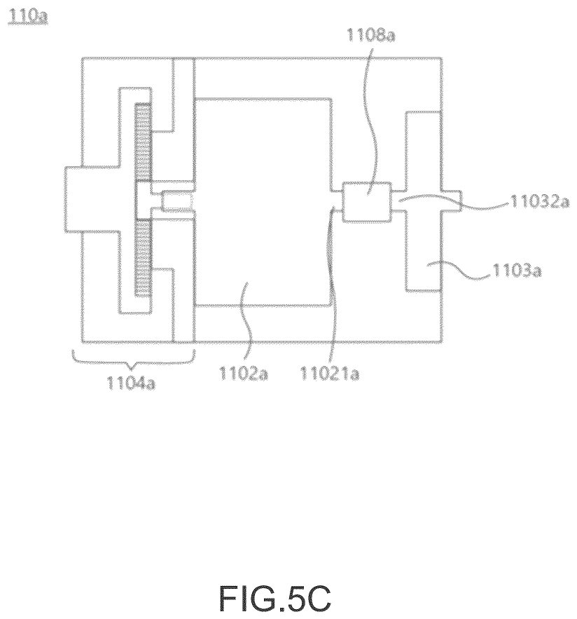

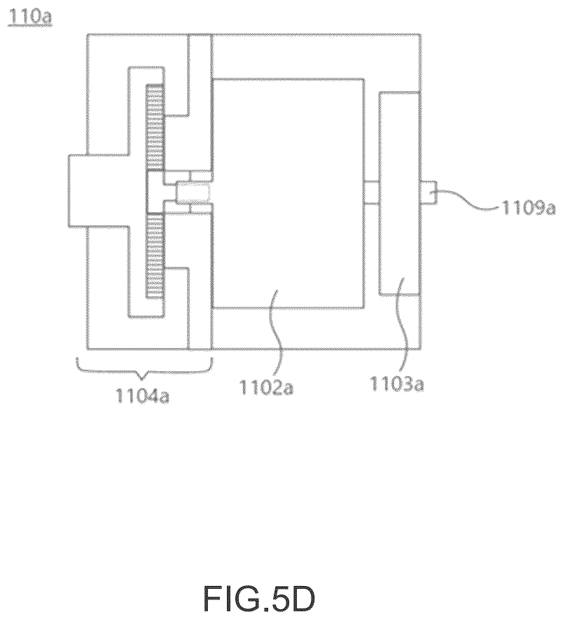

FIG. 5A to FIG. 5D are diagrams illustrating a configuration of arm joint(s) using a capacitive encoder, according to example implementations of the present disclosure. In implementations described with reference to FIGS. 5A to 5D, arm joint 110a is used as examples for description purposes. However, it should be understood that the descriptions do not limit the specific positions of the arm joint 110a within the arm 10. The arm joint 110a may be, for example, a rotary joint.

In some implementations, an arm joint using a capacitive encoder may include a rotating shaft, a brake, and an encoder. In some implementations, the arm joint may further include a reducer, and the rotating shaft, brake, encoder, and reducer may be, for example, coaxially assembled.

Referring to FIG. 5A, in some implementations, the brake 1102a within the arm joint 110a may be connected to a reducer 1104a, and the capacitive encoder 1103a may be sleeved onto the output shaft end 11041a of the reducer 1104a. Advantageously, this configuration may reduce measurement errors of the encoder 1103a that are caused by the transmission mechanism.

In some implementations, the capacitive encoder 1103a may be positioned on the output shaft end 11021a of the brake 1102a.

Referring to FIG. 5B, in some implementations, the reducer 1104a within the arm joint 110a may be connected to one side of the brake 1102a, and the capacitive encoder 1103a may be connected to the opposite side of the brake 1102a. The capacitive encoder 1103a may be hollow and sleeved onto the output shaft 11021a of the brake 1102a.

Referring to FIG. 5C, in some implementations, the reducer 1104a within the arm joint 110a may be connected to one side of the brake 1102a, and the capacitive encoder 1103a may be connected to the opposite side of the brake 1102a. The output shaft 11021a of the brake 1102a may be connected to the output shaft 11032a of the encoder 1103a via a coupling 1108a.

Referring to FIG. 5D, in some implementations, both the brake 1102a and the capacitive encoder 1103a are of hollow design. In such a design, the reducer 1104a within the arm joint 110a may be connected to one side of the brake 1102a, and the capacitive encoder 1103a may be connected to the opposite side of the brake 1102a. The brake 1102a and the capacitive encoder 1103a may be connected via a long shaft 1109a, thus eliminating the need for a coupling and reducing the axial length of the arm joint 110a.

Referring back to FIG. 3, by using outputs of the encoders corresponding to the arm joints 110a, 110b, 110c, 110d, 110e of the arm 10, the relative (e.g., spatial) relationship between two points (e.g., the terminal end 102 and the base end 101) of the arm 10 may be determined, for example, by using the rotational angle of each arm joint, as sensed by the encoders, the mechanical parameters (e.g., sizes and/or configurations of each component) of the arm 10, and/or forward kinematics.

In some implementations, the relative relationship may include positional information and/or angular information. In other words, the relative relationship may include six degrees of freedom. It should be noted that, for the sake of brevity, unless otherwise noted, the present disclosure will describe implementations/examples using positional information only.

In some implementations, the relative relationship between the terminal end 102 and the base end 101 of the arm 10 may be represented by a position/coordinate with respect to a first coordinate system that, for example, taking the base end 101 as a reference point or an origin. In some implementations, the first coordinate system may be a 3D coordinate system such as a Euclidean coordinate system, a spherical coordinate system, etc.

Referring back to FIG. 3, in some implementations, the terminal end 102 may be located on an end of the end effector module 110f of the arm 10 and the terminal adapter structure 12 may be connected at the terminal end 102. In some implementations, the terminal adapter structure 12 may be considered to be part of the end effector module 110f, as described above.

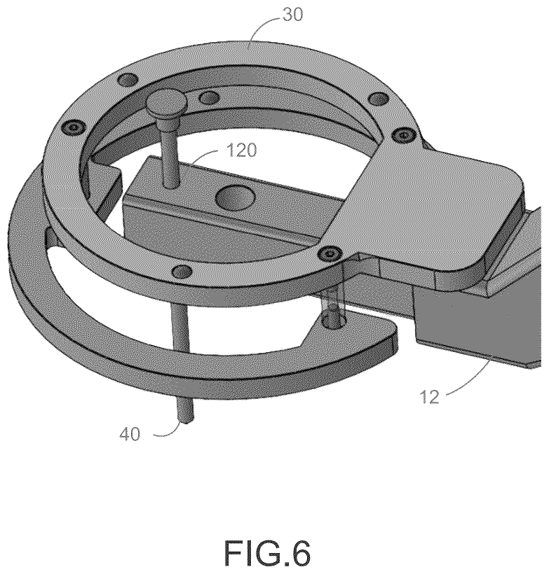

FIG. 6 is a diagram illustrating a terminal adapter structure, according to an example implementation of the present disclosure.

Referring to FIG. 6, the terminal adapter structure 12 may be configured to couple a calibration device 30 and a (surgical) instrument 40.

In some implementations, the terminal adapter structure 12 may include a first structure (not shown) and a second structure 120, where the first structure may be configured to detachably connect the calibration device 30 and the second structure 120 may be configured to detachably connect, secure, and/or limit the instrument 40. In some examples, the first structure may be a connector. In some examples, the second structure may include at least one of a connector, a fixture, and a limiter (e.g., linear motion).

In some implementations, the terminal adapter structure 12 may include the first structure for connecting the calibration device 30 and the calibration device 30 may include the second structure 120 for connecting or limiting the instrument 40.

In some implementations, based on the mechanical parameters of the end effector module, the terminal adapter structure 12 and the calibration device 30/instrument 40, the position/coordinate of any point on the calibration device 30/instrument 40 with respect to the first coordinate system may be determined, based on the method described above.

In some implementations, the (surgical) instrument 40 may be used to perform surgeries and may comprise at least one of a manual or powered surgical tool.

In some implementations, the second structure 120 used to connect/secure/limit the instrument 40 may include a set of sleeves, which may be a single-size sleeve or an expandable sleeve with an adjustable aperture to accommodate the instrument 40 that may have different sizes or shapes. The sleeve set may secure the instrument 40 by using any form of fixation. The sleeve set may further possess features to limit the movement of the instrument 40.

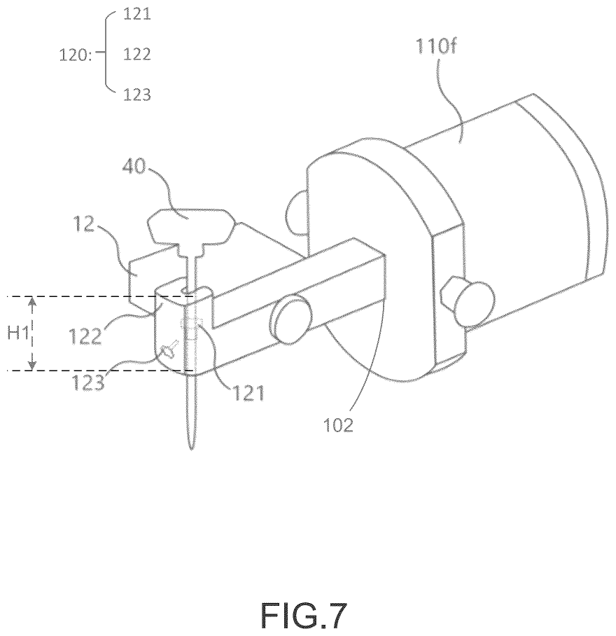

FIG. 7 is a diagram illustrating a structure configured to connect/secure/limit the instrument, according to an example implementation of the present disclosure.

Referring to FIG. 7, the end effector module 110f of the arm 10 may connect to the terminal adapter structure 12, which may include the second structure 120. The second structure 120 may include a sleeve set 121, a limiting mechanism 122, and a clamping mechanism 123. The sleeve set 121 may be configured to secure the instrument 40. The clamping mechanism 123 may enable users to quickly clamp or remove the instrument 40, thus allowing for swift transitions to general surgical modes without using the arm 10. The limiting mechanism 122 may be used to limit the axial depth of the instrument 40. For example, the limiting mechanism 122 may take various forms such as screw locking, magnetic attachment, telescopic adjustment, quick-release, snap-fit, and/or monolithic designs.

In some implementations, a height H1 of the limiting mechanism 122 may be adjustable. By adjusting the height H1, an axial limitation depth of the instrument 40 may be modified. The height H1 of the limiting mechanism 122 may be adjusted by: swapping different heights of limiting mechanisms, using a limiting mechanism with telescopic heights, or stacking multiple limiting mechanisms. In some implementations, to facilitate the assembly/disassembly of the limiting mechanism 122 and the instrument 40, a side of the limiting mechanism 122 may feature a slot that allows for quick installation/removal of the instrument 40.

Advantageously, when the user is operating the instrument 40 during a surgery or an operation, the instrument 40 will not deviate from the angles allowed by the second structure 120, nor will the instrument 40 exceed the range set by the second structure 120.

In some implementations, the second structure 120 may further include a slide rail. The slide rail may be only allowed to slide once the arm joints of the arm 10 are locked, thus ensuring that the instrument 40 can move axially along the aligned target path. At this point, the axial depth of the instrument 40 may be adjusted via the slide rail. By further incorporating a depth calculation mechanism, the depth of the slide rail movement may be calculated. The depth calculation mechanism may include, for example, an encoder or a grating structure on the slide rail.

In some implementations, the calibration device 30 may be used for an image correction to determine a transformation relationship of a 3D coordinate system and a coordinate system of the image(s) that are captured by the medical imaging system 2. Specifically, first position(s)/coordinate(s) of specific point(s) (e.g., on the calibration device 30) in space may be represented with respect to the first coordinate system, as described above. In a case that the specific point(s) are within a field of view of the medical imaging system 2, second position(s)/coordinate(s) of the specific point(s) in the image(s) that captured by the medical imaging system 2 may be represented with respect to a second coordinate system that describes the image space of the image(s). The transformation relationship between the first coordinate system and the second coordinate system may be determined based on the first position(s)/coordinate(s) and the second position(s)/coordinate(s), for example, in a form of transformation matrix. In such a case, any first position/coordinate with respect to the first coordinate system may be transformed into a second position/coordinate with respect to the second coordinate system based on transformation relationship. In some implementations, the second coordinate system may be a 2D coordinate system such as a Cartesion coordinate system, a polar coordinate system, etc. In some implementations, the second coordinate system may be a 3D coordinate system such as a Euclidean coordinate system, a spherical coordinate system, etc.

In some implementations, for the purpose of the image correction as described above, the calibration device 30 may include a certain feature for being captured by the medical imaging system 2. In some examples, the certain feature may be one or more marker(s).

In some implementations, at least two markers on the calibration device 30 may have different absorption rate for a radiation (e.g., X-ray) that is used by the medical imaging system 2. In some implementations, at least two positions within one (each) of the marker(s) may have different absorption rates for the radiation that is used by the medical imaging system 2. Advantageously, when the image that is captured by the medical imaging system 2 includes both the calibration device 30/marker(s) and the target area (e.g., area of lesion, anatomical structure, and/or anatomical), not all marker(s) will merge with the target area (such as bones) image and become difficult to distinguish.

In some implementations, the marker(s) may be non-coplanar, thus forming a control volume. For example, the markers may be located on at least two different planes, e.g., including a first plane and a second plane. In some examples, the first plane and the second plane may be parallel to each other.

In some implementations, the calibration device 30 may include four or five markers. In some implementations, the calibration device 30 may include at least six markers. In some examples, in order to satisfy the algorithmic requirements for the image correction, at least one marker may be non-coplanar with the other markers.

In some implementations, at least three markers may be on the first plane and at least three markers may be on the second plane. In some implementations, the at least three markers on the first plane may define a first ring on the first plane and the at least three markers on the second plane may define a second ring on the second plane.

In some implementations, materials forming the marker(s) may be imageable in the images that are captured by the medical imaging system 2. For example, the marker(s) may be made of materials with high absorption rates for the radiation of the medical imaging system 2 (e.g., such as metals or ceramics for an X-ray imaging system).

In some implementations, the marker(s) on the calibration device 30 may have known relative relationships and geometric characteristics. Therefore, the position(s) (e.g., with respect to the first coordinate system) of the marker(s) may be known or determined when the calibration device 30 is connecting to the terminal adapter structure 12, based on the method described above.

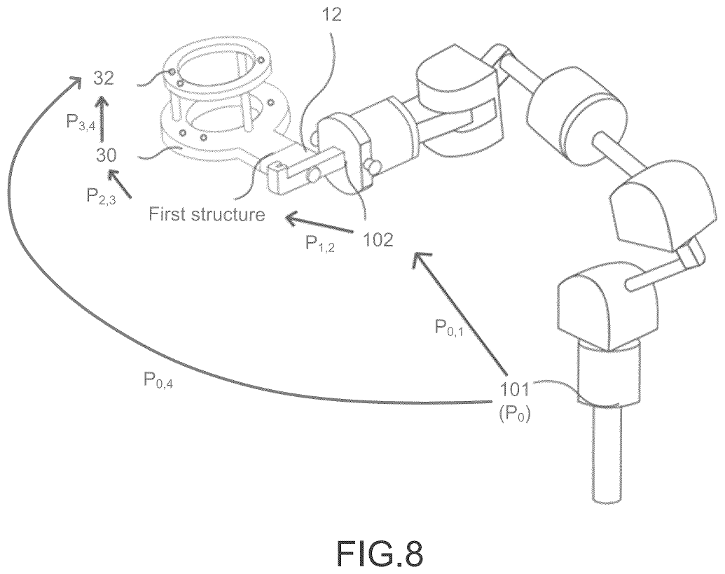

FIG. 8 is a diagram illustrating a determination of positions of the markers, according to an example implementation of the present disclosure.

Referring to FIG. 8, when the calibration device 30 connects to the terminal adapter structure 12, positions P_{0,4} of the markers 32 with respect to the first coordinate system (e.g., which takes the position P₀ of the base end 101 as a reference or an origin) may be determined.

For example, with respect to the first coordinate system, a position P_{0,1} of the terminal end 102 may be determined based on outputs of the encoders corresponding to the arm joints 110a, 110b, 110c, 110d, 110e of the arm 10, the mechanical parameters (e.g., sizes and/or configurations of each component) of the arm 10, and/or forward kinematics, as described above. Additionally, the relative position P_{1,2} of the first structure with respect to the terminal end 102 may be determined according to known parameters (e.g., sizes and/or configurations) of the terminal adapter structure 12, the relative position P_{2,3} of a specific point of the calibration device 30 with respect to the first structure may be determined according to known parameters of the calibration device 30, and the relative positions P_{3,4} of the markers 32 on the calibration device 30 with respect to the specific point of the calibration device 30 may be determined according to known parameters of the calibration device 30. In such a case, the positions P_{0,4} of the markers 32 with respect to the first coordinate system may be determined based on the positions P_{0,1}, P_{1,2}, P_{2,3}, and P_{3,4}.

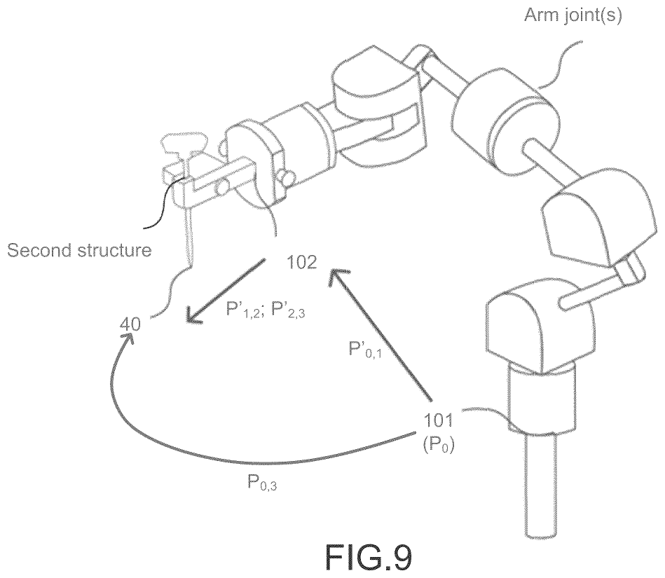

FIG. 9 is a diagram illustrating a determination of a position of an instrument, according to an example implementation of the present disclosure.

Referring to FIG. 9, when the instrument 40 connects/secures to the terminal adapter structure 12, the position(s) P′_{0,3} of the instrument 40 (e.g., including a position of a tip of the instrument 40) with respect to the first coordinate system (e.g., which takes the position P₀ of the base end 101 as a reference or an origin) may be determined.

For example, with respect to the first coordinate system, a position P′_{0,1} of the terminal end 102 may be determined based on outputs of the encoders corresponding to the arm joints 110a, 110b, 110c, 110d, 110e of the arm 10, the mechanical parameters (e.g., sizes and/or configurations of each component) of the arm 10, and/or forward kinematics, as described above. Additionally, the relative position P′_{1,2} of the second structure 120 with respect to the terminal end 102 may be determined according to known parameters (e.g., sizes and/or configurations) of the terminal adapter structure 12, and the relative position(s) P′_{2,3} of the instrument 40 (e.g., including a position of a tip of the instrument 40) with respect to the second structure 120 may be determined according to known parameters of the instrument 40 and/or the second structure 120. In such a case, the position(s) P′_{0,3} of the instrument 40 with respect to the first coordinate system may be determined based on the positions P′_{0,1}, P′_{1,2}, and P′_{2,3}.

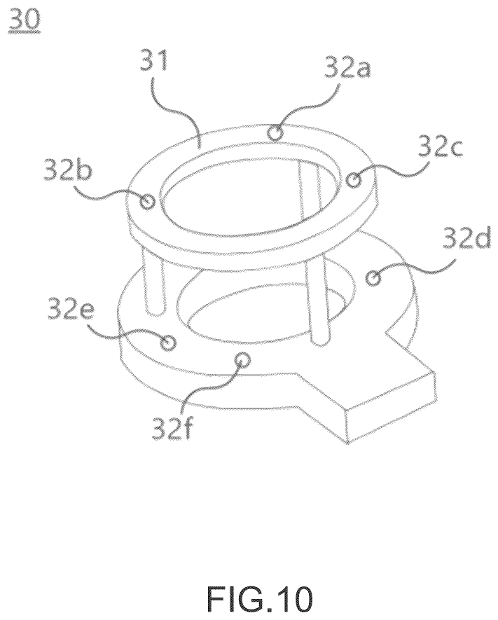

FIG. 10 is a diagram illustrating a calibration device, according to an example implementation of the present disclosure.

Referring to FIG. 10, the calibration device 30 may include a structural framework 31 and multiple (e.g., six) markers 32a, 32b, 32c, 32d, 32e, 32f. The structural framework 31 may include a first ring on a first plane and a second ring on a second plane different from and parallel to the first plane. The markers 32a, 32b, 32c may be located on the first ring and the markers 32d, 32c, 32f may be located on the second ring.

In some implementations, the first ring and the second ring may be concentric. In such a case, based on the markers 32a, 32b, 32c, 32d, 32e, 32f in a 2D image, two rings may be determined, and the centers of the two rings may be used to determine an axial direction (e.g., in a 3D space). In some examples, the relative relationship between the calibration device 30, instrument 40, and the terminal adapter structure 12 may be arranged as shown in FIG. 6, and in such a case, the axial direction that is determined by the centers of the two rings may also be used for determining the orientation of the instrument 40.

In some implementations, sizes of the first ring and the second ring may be the same. In other words, the first ring and the second ring may define a hollow cylinder.

In some implementations, sizes of the first ring and the second ring may be different. In other words, the first ring and the second ring may define a hollow circular frustum.

In some implementations, material of the structural framework 31 may have a lower absorption rate, for the radiation used by the medical imaging system 2, than that of all or at least one of the marker(s). In such a case, the two rings may be determined from the 2D image without detection and calculation using the marker(s).

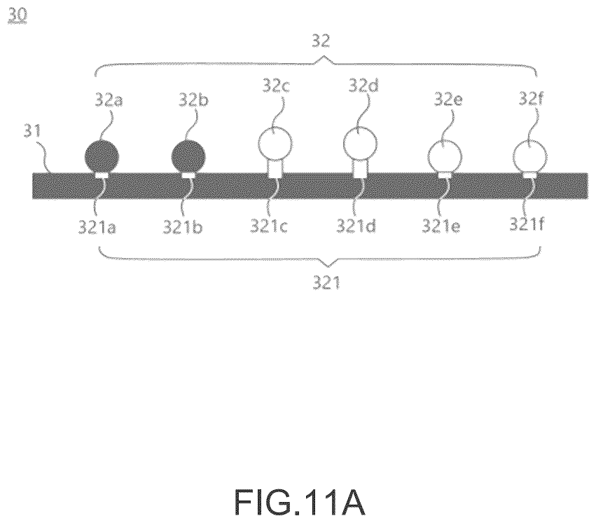

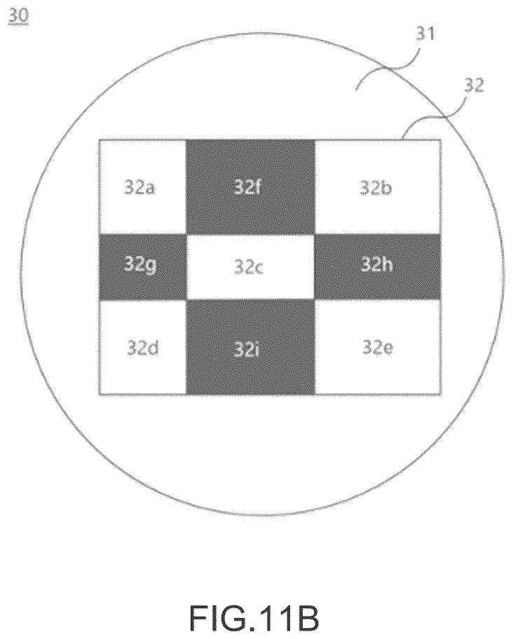

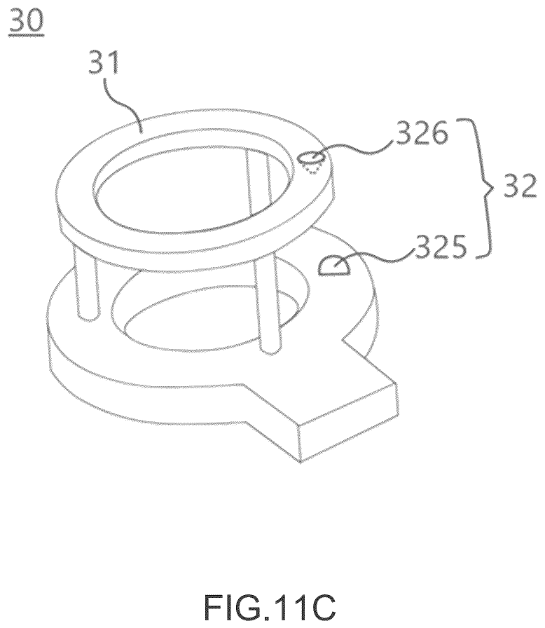

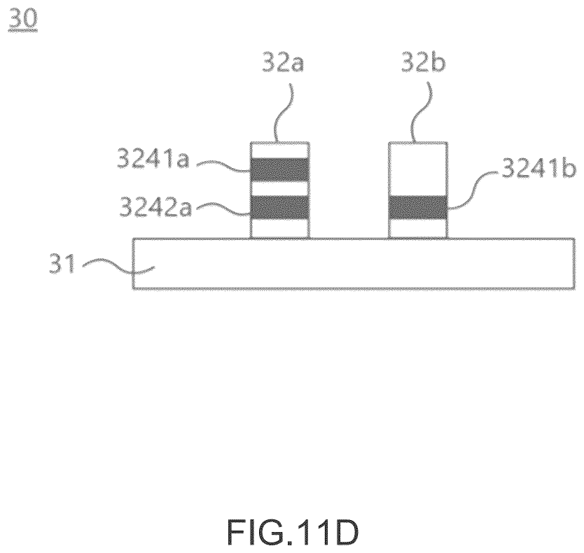

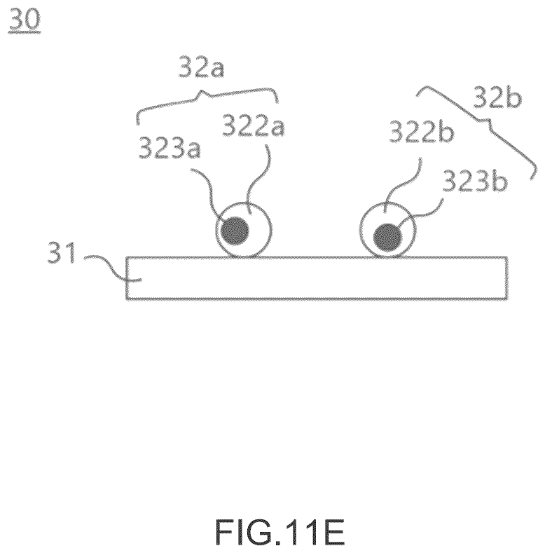

FIG. 11A to FIG. 11E are diagrams illustrating markers on the calibration device, according to example implementations of the present disclosure.

In some implementations, at least two of the markers may have different absorption rates for the radiation used by the medical imaging system 2 (e.g., X-ray). Advantageously, such configuration may allow the markers and the target area to avoid interfering with each other, making both the markers and the target area identifiable in the image, and thus facilitating easier image recovery, enhancement, sharping and denoising. Advantageously, using multiple markers with varying absorption rates may enable the identification of each marker in the medical image, thus improving efficiency in image correction.

In some implementations, different absorption rates may be implemented by fillable marker(s). In some examples, all or at least one of the markers may be implemented as fillable.

Referring to FIG. 11A, the calibration device 30 may include the structural framework 31 and the markers 32a, 32b, 32c, 32d, 32e, 32f each including a housing (e.g., at least partially transparent or translucent for the radiation) and a gate 321a, 321b, 321c, 321d, 321e, 321f. Each marker 32a, 32b, 32c, 32d, 32c, 32f may be filled with a contrast agent to enhance visibility in the image(s) that is/are captured by the medical imaging system 2, that is controlled by pumps through the gates 321a, 321b, 321c, 321d, 321e, 321f. Advantageously, users may choose marker(s) to be filled to avoid interfering with the identification of target area (e.g., based on a pre-captured image). In the example shown in FIG. 11A, the markers 32a, 32b are filled with the contrast agent, while the markers 32c, 32d, 32c, 32f are not filled with the contrast agent.

In some implementations, the markers may be arranged as a grid.

Referring to FIG. 11B, the calibration device 30 may include the structural framework 31 and the feature 32. The feature 32 may include multiple markers 32a, 32b, 32c, 32d, 32e, 32f, 32g, 32h, 32i, each being a known-size cell of a grid. Adjacent cells/markers may have different absorption rates for the radiation used by the medical imaging system 2. For instance, the cells/markers 32f, 32g, 32h, 32i may have higher absorption rates than the cells/markers 32a, 32b, 32c, 32d, 32c. For instance, the cells/markers 32f, 32g, 32h, 32i may be absorbent, while the cells/markers 32a, 32b, 32c, 32d, 32e may be non-absorbent. In such a case, with the known differences in absorption rates among the cells/markers, the image contrast may be enhanced by image post-processing. Additionally, via image processing, the center points of each cell/marker may be extracted as coordinate points for the image correction.

In some implementations, different absorption rates may be implemented by geometric differences.

Referring to FIG. 11C, the calibration device 30 may include the structural framework 31 and the markers 325, 326. The marker 325 may be formed on the structural framework 31 as a protruding structure (e.g., in a shape of a cylinder, sphere, or hemisphere, etc.). On the other hand, the marker 326 may be formed on the structural framework 31 as a recessed structure (e.g., in a shape of a cylinder, sphere, or hemisphere, etc.). In such a case, the geometric differences between markers 325 and 326 may create contrast in the images (e.g., X-ray images) between these markers 325, 326 and the structural framework 31. In some implementations, the markers 325, 326 and the structural framework 31 may be made of materials that create contrast differences in the images. For example, the markers 325, 326 may be tungsten steel balls, and the structural framework 31 may be an aluminum alloy frame.

In some implementations, the different absorption rates may be implemented by multilayer structures. In some implementations, layers in the multilayer structure may be adjustable.

Referring to FIG. 11D, the calibration device 30 may include the structural framework 31 and the markers 32a, 32b, and each of the markers 32a, 32b may include a multilayer structure. Specifically, each of the markers 32a, 32b may include one or more imaging layers (e.g., each having the same absorption rate). For example, the marker 32a may have two imaging layers 3241a, 3242a, while the marker 32b may have one imaging layer 3241b, such that the marker 32a may have a higher absorption rate than the marker 32b. In some implementations, the imaging layer(s) within the multilayer structure of the marker may be adjustable. Users may adjust the composition of the imaging layers based on the conditions of the image that is captured by the medical imaging system 2. For example, when the image contrast is poor or the markers 32a, 32b obstruct the visibility of patient anatomical features in the image, the number of imaging layers in the markers 32a, 32b may be adjusted (e.g., added or reduced) to enhance image quality.

In some implementations, for one or each of the markers, at least two positions therein may have different absorption rates for the radiation used by the medical imaging system 2. Advantageously, such configuration may ensure that each marker is at least partially visible in the image that is captured by the medical imaging system 2.

In some implementations, the marker(s) having two positions with different absorption rates may be implemented by composite materials.

Referring to FIG. 11E, the markers 32a may include a larger sphere 322a with a smaller sphere 323a that is embedded within the larger sphere 322a. The spheres 322a, 323a may have distinct absorption rates and may be imaged in the images (e.g., X-ray images). From another perspective, the two spheres 322a, 323a may be considered to be distinct markers.

Referring to FIG. 11E, the markers 32b may include a larger sphere 322b with a smaller sphere 323b that is embedded within the larger sphere 322b. The sphere 323b may form a hollow interior within the sphere 322b, such that the spheres 322b, 323b may have distinct absorption rates. From another perspective, the two spheres 322b, 323b may be considered to be distinct markers.

Advantageously, the space occupied by the markers (e.g., 322a, 322b, 323a, and 323b) may be reduced, thus reducing the obstruction of patient anatomical features in the images that is caused by the markers.

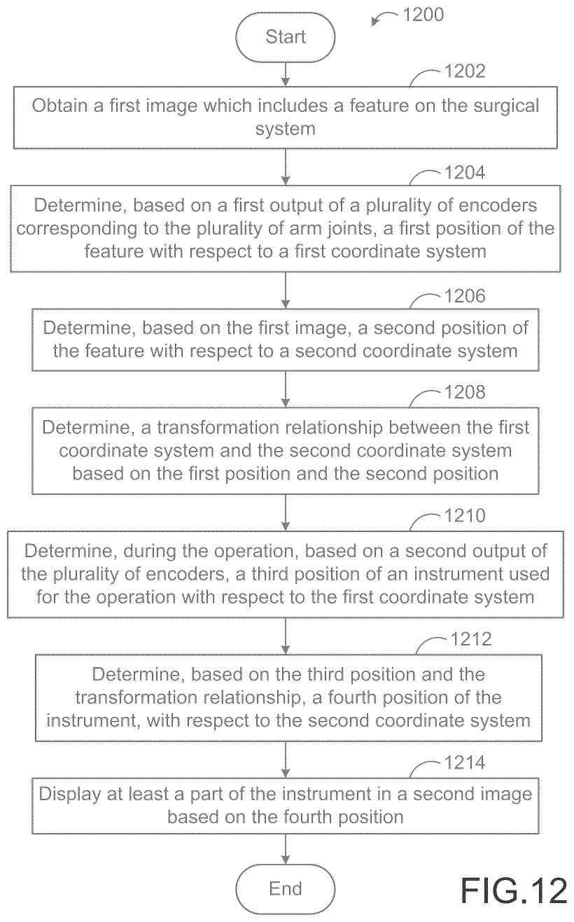

FIG. 12 is a flowchart illustrating a method/process for navigating a surgical operation, according to an example implementation of the present disclosure. In some implementations the process 1200 may be performed by the navigation system 20, in cooperation with the arm 10 described with reference to FIGS. 1 to 11. It should be noted that although actions 1202, 1204, 1206, 1208, 1210, 1212, and 1214 are illustrated as separate actions represented as independent blocks in FIG. 12, these separately illustrated actions should not be construed as necessarily order-dependent. Unless otherwise indicated, the order in which the actions are performed in FIG. 12 is not intended to be construed as a limitation, and any number of the disclosed blocks may be combined in any order to implement the method, or an alternate method. Moreover, each of actions 1202, 1204, 1206, 1208, 1210, 1212, and 1214 may be performed independently of other actions and may be omitted in some implementations of the present disclosure.

In some implementations, a (surgical) operation may be performed by using the arm 10 and the (surgical) instrument 40, and the navigation system 20 may be used to navigate the operation by presenting/displaying guidance information related to the instrument 40, e.g., including the spatial positioning, appearance style, and/or movement state of the instrument 40. For example, the navigation system 20 may show a medical image of the patient along with a virtual surgical instrument, and the position of the virtual surgical instrument in the medical image may move in accordance with the actual movements of the instrument 40 in space.

Referring to FIG. 12, in action 1202, the process 1200 may start by the navigation system 20 obtaining a first image which includes a feature on the surgical system 1. Specifically, the first image may include a medical image, and the medical image may be captured by the medical imaging system 2. The processor 22 may obtain the first image from the medical imaging system 2, or through a reshooting process, which is not limited in the present disclosure.

In some implementations, the feature in the first image may be the markers 32 on the calibration device 30, as described above. Specifically, when performing the action 1202, the user may ensure that the calibration device 30 is connected to the terminal adapter structure 12, and the markers 32 (e.g., 4, 5, 6, or more than 6 markers) on the calibration device 30 are located within the field of view of the medical imaging system 2.

In some implementations, the first image may further include a target area of the patient. For example, when taking the medical image using the medical imaging system 2, the user may align all or at least a part of the target area for surgery through the window that is formed by the markers 32 on the calibration device 30. Consequently, in the medical image, multiple markers 32 may surround all or at least a part of the target area targeted for surgery.

Referring back to FIG. 6, in some implementations, the first structure and the second structure 120 may be configured such that when the first structure connects the calibration device 30 and the second structure 120 connects/secures to the instrument 40, in the first image the markers 32 may surround the position of at least part (e.g., a tip or a front end) of the instrument 40. As subsequent needs may arise to predict, in an image, the position of the at least part of the instrument 40 or its relative position to the target area, such a configuration may yield more accurate predictions of the position of the at least part of the instrument 40.

In action 1204, the navigation device 20 may determine, based on a first output of a plurality of encoders that correspond to the plurality of arm joints 110a, 110b, 110c, 110d, 110e, a first position of the feature with respect to a first coordinate system.

Specifically, the processor 22 may determine the first position(s)/coordinate(s) of the markers 32 with respect to the first coordinate system using outputs of the encoders that correspond to the arm joints 110a, 110b, 110c, 110d, 110e, based on the method described above.

In action 1206, the navigation device 20 may determine, based on the first image, a second position of the feature with respect to a second coordinate system.

In some implementations, in a case that the markers are within a field of view of the medical imaging system 2 when taking the medical image within the first image, the second position(s)/coordinate(s) of the markers in the first image may be represented with respect to the second coordinate system that describes the image space of the first image. As such, the processor 22 may determine the second position(s)/coordinate(s) based on the first image, for example, by defining the second coordinate system and performing an image recognition on the first image.

In action 1208, the navigation device 20 may determine a transformation relationship between the first coordinate system and the second coordinate system based on the first position and the second position.

In some implementations, the transformation relationship may be a transformation matrix that maps position(s)/coordinate(s) in the first coordinate system to position(s)/coordinate(s) in the second coordinate system.

In some implementations, the number of the markers may be 6 or more than 6. In such a case, the transformation relationship may be determined based on the first positions and the second positions of the six or more markers with respect to the first coordinate system and the second coordinate system.

In some implementations, the number of the markers may be 4 or 5. In such a case, additional information such as intrinsic parameters of the medical imaging system 2 may be needed for determining the transformation relationship. In other words, the transformation relationship may be determined based on the intrinsic parameters of the medical imaging system 2 (e.g., preset to the processor 22), in addition to the first positions and the second positions of the four or five markers with respect to the first coordinate system and the second coordinate system.

In some implementations, the determination of the transformation relationship may be performed by the processor 22 using methods such as Direct Linear Transformation (DLT), Perspective-n-Point (PnP), bundle adjustment, etc.

Through actions 1202 to 1208, the current surgical environment, including the patient, the arm 10, the coordinate systems needed for describing the current surgical environment, and the transformation relationship between the coordinate systems, may be well-prepared. Based on the established transformation relationship, any point that can be represented with respect to the first coordinate system may be transformed into the second coordinate system.

In action 1210, the navigation device 20 may determine, during the operation, based on a second output of the plurality of encoders, a third position of an instrument 40 used for the operation with respect to the first coordinate system.

In some implementations, after the first and second coordinate systems needed for describing the current surgical environment, and the transformation relationship between the first and second coordinate systems are well-prepared, the user may start to perform the surgical operation by using the instrument 40 that is connected to/secured to/limited by the terminal adapter structure 12.

During the operation, third position(s)/coordinate(s) of the instrument 40 (e.g., any points on the instrument 40, including the tip) with respect to the first coordinate system may be determined by the processor 22 using outputs of the encoders that correspond to the arm joints 110a, 110b, 110c, 110d, 110e, based on the method described above.

In action 1212, the navigation device 20 may determine, based on the third position and the transformation relationship, a fourth position of the instrument 40, with respect to the second coordinate system.

Specifically, fourth position(s)/coordinate(s) of the instrument 40 (e.g., any points on the instrument 40, including the tip) with respect to the second coordinate system may be determined by the processor 22 based on the third position(s)/coordinate(s) of the instrument 40 (e.g., any points on the instrument 40, including the tip) with respect to the first coordinate system and the transformation relationship.

In some implementations, the transformation relationship may be represented as a transformation matrix, such that the transformation matrix can be applied to the third position(s)/coordinate(s) to obtain the fourth position(s)/coordinate(s).

In action 1214, the navigation device 20 may display at least a part of the instrument 40 in a second image based on the fourth position. Then, the process 1200 may end.

Specifically, based on the ability of the processor 22 to determine the fourth position(s)/coordinate(s) of (e.g., all points of) the instrument 40 in the image space with respect to the second coordinate system, the processor 22 may display the tip or a specific length (e.g., due to hardware/software constraints or any other requirements) of the front end of the instrument 40 in the second image through the output device 21, based on the fourth position(s)/coordinate(s) with respect to the second coordinate system.

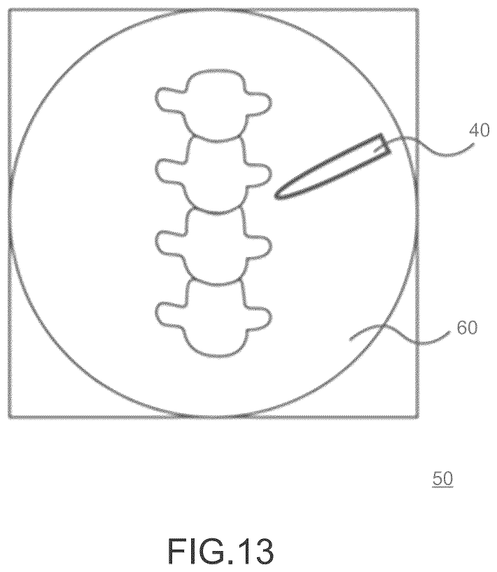

FIG. 13 is a diagram illustrating an output image, according to an example implementation of the present disclosure.

Referring to FIG. 13, in some implementations, the second image 50 may depict (e.g., as a predictive rather than an actual, real-time capture by the medical imaging system 2) the at least one part of the instrument 40 and a target area 60 of the patient. To facilitate navigation during the surgery or to provide guidance for the operation, the processor 22, for example, may use the target area 60 as a background a backdrop and (e.g., dynamically) display, in real-time, the current position of the at least one part of the instrument 40 in the second image 50.

In some implementations, the second image 50 may include a superposition of the first image (e.g., which may include the target area 60) and the at least one part of the instrument 40.

Based on the above, the present disclosure, by installing encoders in the arm joints, allows the surgical environment to be accurately positioned with just a single medical image, thus significantly reducing potential radiation exposure for both the surgeon and the patient. Additionally, the designed calibrator may reduce the obstruction of patient anatomical features in the images that is caused by the markers.

In view of the present disclosure, various techniques may be used for implementing the disclosed concepts without departing from the scope of those concepts. Moreover, while the concepts have been disclosed with specific reference to certain implementations, a person of ordinary skill in the art may recognize that changes may be made in form and detail without departing from the scope of those concepts. As such, the disclosed implementations are considered in all respects as illustrative and not restrictive. It should also be understood that the present disclosure is not limited to the specific implementations disclosed. Still, many rearrangements, modifications, and substitutions are possible without departing from the scope of the present disclosure.

What is claimed is:

1. A calibration device for a medical imaging system, comprising:
a structural framework; and
a plurality of markers arranged on the structural framework,
wherein:
at least two of the plurality of markers are with different absorption rates for a radiation used by the medical imaging system, or
at least two positions within one of the plurality of markers are with different absorption rates for the radiation.

2. The calibration device of claim 1, wherein a number of the plurality of markers is greater than or equal to 4.

3. The calibration device of claim 1, wherein
one of the plurality of markers comprises a multilayer structure,
the multilayer structure comprises at least one imaging layer, and
a number of the at least one imaging layer is adjustable in the multilayer structure.

4. The calibration device of claim 1, wherein
one of the plurality of markers comprises a first sphere and a second sphere,
the second sphere is embedded in the first sphere, and
the first sphere and the second sphere have different absorption rates for the radiation.

5. The calibration device of claim 1, wherein
one of the plurality of markers comprises a first sphere and a second sphere, and
the second sphere forms a hollow interior of the first sphere.

6. The calibration device of claim 1, wherein one of the plurality of markers comprises:
a housing at least partially transparent for the radiation; and
a gate configured to allow a contrast agent to flow into the housing.

7. The calibration device of claim 1, wherein
the plurality of markers comprises a grid with a plurality of cells, and
adjacent cells of the plurality of cells have different absorption rates for the radiation.

8. The calibration device of claim 1, wherein
a first marker of the plurality of markers comprises a protrusion structure on the structural framework, and
a second marker of the plurality of markers comprises a recessed structure on the structural framework.

9. The calibration device of claim 1, wherein the structural framework comprises:
a first ring located on a first plane,
a second ring located on a second plane parallel to the first plane, and
at least three of the plurality of markers are located at the first ring and at least three of the plurality of markers are located at the second ring.

10. The calibration device of claim 9, wherein the second ring is concentric with the first ring.

11. The calibration device of claim 9, wherein the first ring and the second ring define a hollow circular frustum or a hollow cylinder.

12. The calibration device of claim 1, wherein at least one of the plurality of markers is non-coplanar with the other of the plurality of markers.

13. The calibration device of claim 1, wherein the radiation comprises an X-ray.
